(19) United States
(12) Patent Application Publication
(10) Pub. No.: US 2009/0087483 A1
(43) Pub. Date: Apr. 2, 2009

(54) ORAL DOSAGE COMBINATION PHARMACEUTICAL PACKAGING

(76) Inventor: Raymundo A. Sison, Princeton, NJ (US)

Correspondence Address: HAYES SOLOWAY P.C., 3450 E. SUNRISE DRIVE, SUITE 140, TUCSON, AZ, 85718, US

(21) Appl. No.: 11/863,156

(22) Filed: Sep. 27, 2007

(51) Int. Cl. A61K 9/48; A61K 47/26; A61P 3/10

(52) U.S. Cl. 424/451; 514/781

(57) ABSTRACT

Pharmaceutical fixed dose combination products are formed by merging a fixed dose of a first pharmaceutical formulation from primary module, with a fixed dose of a second pharmaceutical formulation from a secondary module. In a preferred embodiment the first and second pharmaceutical formulations are separated from one another in a three piece capsule, a capsule-in-a-capsule or a tablet-in-a-capsule, and the primary and secondary modules are interchangeable.

### BACKGROUND OF THE INVENTION

The present invention relates to the packaging of pharmaceuticals and drugs for medical uses. The invention has particular utility in the packaging of combinations of two or more pharmaceutical formulations or drugs for the same or co-morbid therapy, and will be described in connection with such utility, although other utilities are contemplated.

### DESCRIPTION OF THE PRIOR ART

The convenience of co-administered two or more active pharmaceutical ingredients in a unit dosage form, as opposed to the administration of a number of separate doses of two or more pharmaceuticals at regular intervals, has been recognized in the pharmaceutical arts and is described in prior U.S. Pat. Nos. 6,428,809 and 6,702,683, and co-pending application Ser. Nos. 10/756,124 and 10/479,438 and Provisional Application No. 60/727,029. Advantages to the patient and clinician include (1) minimization or elimination of local and/or systemic side effects; (2) more effective treatment of co-morbid conditions; (3) improved polypharmacy; and (4) better patient compliance with overall disease management, which in turn may lead to reduced costs due to fewer trips to the physician, reduced hospitalization, and improved patient well-being.

While fixed dose combination products, with two or more formulations combined or co-formulated in a single dosage form are useful in multiple drug regimens where improved clinical effectiveness, enhanced patient adherence and simplified dosing are desired. Pharmaceutical drug product development of solid oral dosage forms is complicated at both the R&D level and at the commercial manufacturing level for these products vs. single component products due to various factors. Such factors might include (1) drug-drug interaction, (2) drug-excipient interaction, (3) simultaneous release profiles, (4) differential release profiles, and (5) blend uniformity of each drug component.

Typically, development of fixed dose combination products involve a selection from available dosage forms at an early stage including the following options: 1) single compartment fixed dose combination products such as tablets or capsules containing an intimate mixture of formulated drug product active ingredients, and 2) Multi-compartment fixed dose combination products such as multi-layer compressed tablets, multi-layer coated tablets, multi-particulate systems and multiple compartment systems. Each system has unique formulation development advantages and disadvantages and each system has unique commercial manufacturing advantages and disadvantages.

In the aforesaid U.S. Pat. Nos. 6,428,809 and 6,702,683 there is described packaging two or more active pharmaceuticals or drugs, segregated from one another, in a readily ingestible pharmaceutical delivery package which may take the form of, for example, a tablet or capsule. Various drug combinations are described and claimed in our aforesaid patents.

In parent application Ser. No. 11/549,492 there is provided a fixed dose combination medication delivery package which is simple to manufacture. More particularly, in one embodiment of the parent application, there is provided a pharmaceutical delivery package comprising fixed unit dose quantities of two or more different active pharmaceutical ingredients (a) combined in a single delivery package, and (b) segregated from one another within said package wherein said package comprises a core containing a first active pharmaceutical ingredient surrounded at least in part by a capsule containing a second active pharmaceutical ingredient. The active pharmaceutical ingredient is defined here as either single pharmaceutical ingredient, optionally combined with appropriate excipients, or more than one pharmaceutical ingredient, optionally combined with appropriate excipients. The invention described and claimed in the parent applications provide certain unique and advantageous combinations of drugs that address or overcome one of several issues relating to combinational drug therapy, including more efficient treatment of co-morbid conditions, polypharmacy, reduction of adverse side effects, adjuctive therapy and known drug interactions. In one embodiment, the delivery package is designed to provide for essentially simultaneous release of the two or more pharmaceutical ingredients. In another embodiment, the pharmaceutical delivery package provides for different release rates of the two or more pharmaceutical ingredients, or differential release of the two or more pharmaceutical ingredients.

### SUMMARY OF THE INVENTION

The present application provides improvements over the inventions described and claimed in the aforesaid parent applications.

More particularly, selecting among the current options for fixed dose combination products, a balance between risk and cost are critical to the feasibility of drug product development and manufacture. In other manufacturing industries, such as construction, transportation and packaging, modular design techniques have been applied to leverage efficiencies in standardization with gains created by customization to achieve affordable innovation. These concepts can be applied broadly to pharmaceutical/dietary supplement product development and manufacturing. Specifically, the present invention provides modular design for pharmaceutical packaging employing a unique three (3)-piece capsule delivery system.

Utilizing modular design concepts, the three (3)-piece capsules of the present invention and their method of filling enables those skilled in the art of drug development and manufacture to contain costs and minimize risks by leveraging standardized formulations and processes into innovative fixed dose combination products. With standardized formulations and processes, those skilled in the art of drug development and manufacture can focus their resources on new and customized elements of a formulation and process without the need to reformulate or modify its existing elements.

As used herein the term “fixed dose combination medication delivery package” is one in which two or more drug components or supplements, including vitamins, minerals and phytochemicals are packaged together, isolated from one another in a single dosage form. The drug components may each comprise an active pharmaceutical formulation or ingredient or one of the drug components may comprise an active pharmaceutical formulation or ingredient while the other comprises a substance that effects the other formulation or ingredient, such as, through an acid base reaction, or a substance that potentiates or suppresses the other in a known and predictable manner, or a substance that suppresses or increases absorption time or uptake of the other formulation or ingredient, or a substance that suppresses or increases metabolism through enzymatic activity and effect absorption of the other formulation or ingredient. Also, in yet another embodiment, the pharmaceutical delivery package includes two or more pharmaceutical formulations or ingredients packaged in a manner whereby one or more of the ingredients will be released at different sites within the alimentary canal.

The drug components also may comprise pharmaceuticals as well as supplements including vitamins, minerals, phytochemicals. Thus, as used herein “drugs” and “pharmaceuticals” are intended to include pharmaceutical and drug formulations and ingredients as well as various supplements including vitamins, minerals and phytochemicals.

### BRIEF DESCRIPTION OF THE DRAWINGS

Further features and advantages of the present invention will become clear from the following detailed description taken in conjunction with the accompanying drawings, wherein like numerals depict like parts, and wherein:

FIGS. 1a and 1b diagrammatically illustrate a three (3)-piece capsule combination medication delivery system in accordance with one embodiment of the present invention;

FIGS. 2-4 diagrammatically illustrate three processes utilizing 4 independent modules for the formation of the combination medication delivery system in accordance with other embodiments of the present invention;

FIG. 5 diagrammatically illustrates a bilayered tablet combination medication delivery system made in accordance with the prior art;

FIGS. 6a and 6b diagrammatically illustrate other embodiments of combination medication delivery systems according to the prior art; and

FIG. 7 diagrammatically illustrates yet other prior art process for the formulation combination medication delivery system.

### DETAILED DESCRIPTION OF PREFERRED EMBODIMENTS

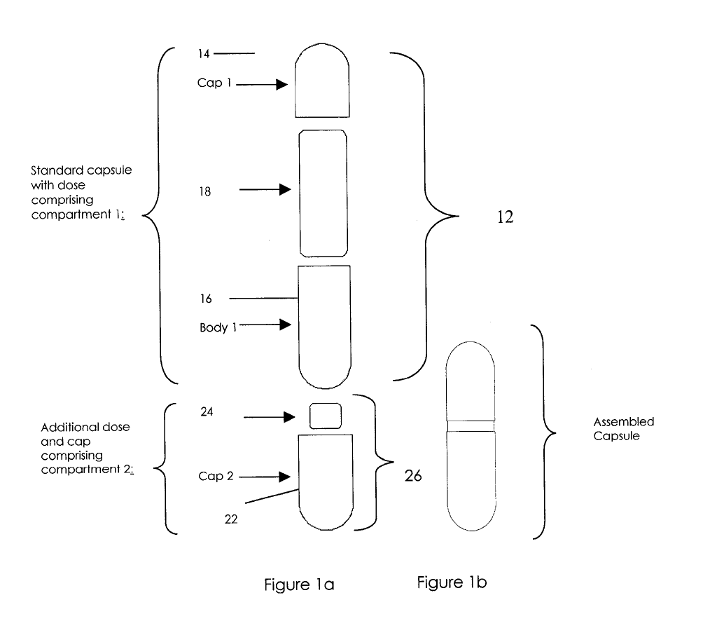

Referring first to FIGS. 1a-1b, there is diagrammatically illustrated the formation of a combination medication delivery system in accordance with one embodiment of the invention. Referring first to FIGS. 1a and 1b, there is illustrated a 3-piece capsule system comprised of two compartments, a first compartment 12 consisting of a two piece capsule 14, 16 for holding a first pharmaceutical formulation 18, and a second compartment 26 that is formed by a second half-capsule or cap 22 for containing a second pharmaceutical formulation 24. Cap 22 is formed to lock onto the body of the first capsule 16. Capsules 14, 16 and 22 preferably, but not necessarily are comprised of hard gelatin.

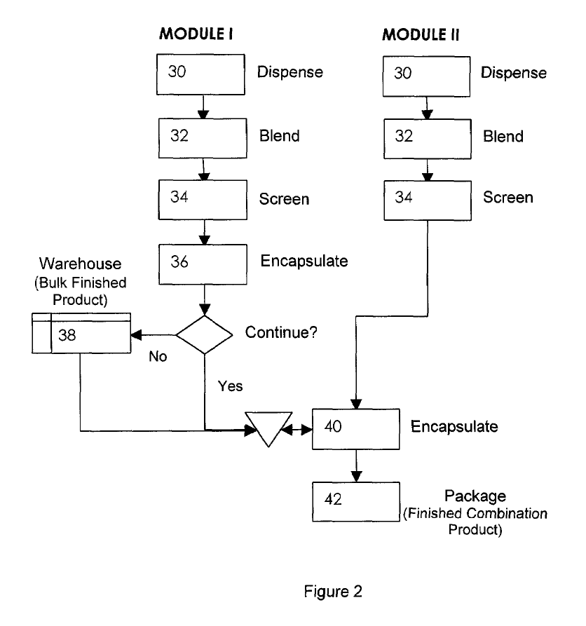

Combination medication delivery system capsules of the present invention may be manufactured utilizing separate and predefined modules that culminate in the filling of compartment 12 and compartment 26. The filling of compartment 12 creates an independent encapsulated finished dosage form; the filling of compartment 26 creates a fixed dose combination package dosage form when appended to the compartment 12 capsule. Each module comprises several defined unit operations. Referring to FIG. 2 Module I includes the step of dispensing 30, blending 32, screening 34 and encapsulation 36. The bulk product can be warehoused 38 before further processing or packaging in part or whole or distributed as a stand- above product. Alternatively, the bulk product formed in Module I can be merged immediately in part or whole with an encapsulation step 40 (Module II) to form the finished fixed dose combinational product lot 42.

The two processes, i.e. Modules I and II are merged in the final encapsulation and packaging unit operations.

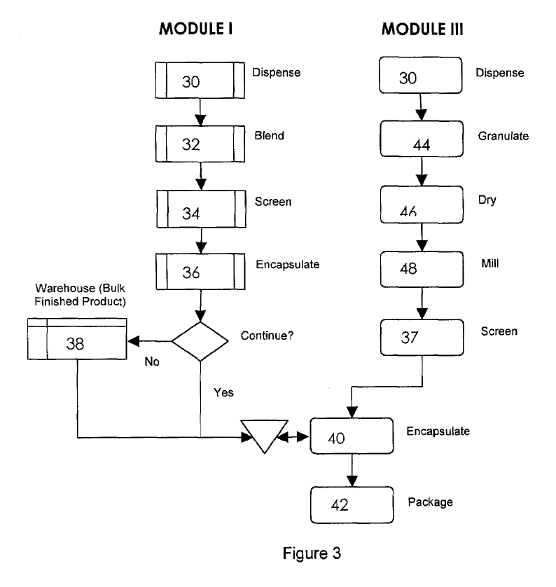

Referring also to FIG. 3, once the process or processes are determined (Module I), modular design allows interchangeability of modules, so that, for example an alternative process e.g. including granulation 44, drying 46 and milling 48 (Module III) to be coupled with a predefined process

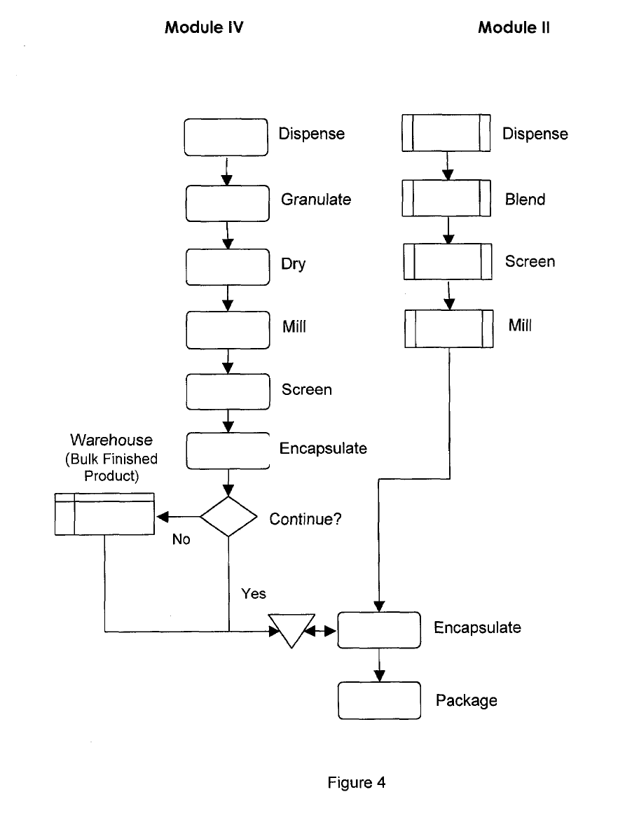

By developing a set of predefined modules, various combinations are possible without the need for a unique and extensive development program for each combination. For example, as shown in FIG. 4, the predefined process Module II, may be combined with a new module (Module IV) to create a unique fixed dose combination. Utilizing formulation and process modules, the standardized Module II allows an R&D and commercial manufacturing unit to focus their resources on Module IV development.

A feature and advantage of the present invention and their methods of manufacture are in the level of modular granularity, the flexibility in designing formulations/processes and simplicity of substituting the modules in order to create various and novel fixed dose combinations. Alternatives for preparing combinational doses are not entirely satisfactory. These include monolithic dose forms, and compartmentalized dosage forms, capsule in capsule, tablet in capsule and multi-unit combination drug systems.

### Monolithic Dosage Forms

Monolithic dosage forms do not employ modular design concepts on the level of granularity as described here. Intimate mixtures are created for each unique fixed dose combination formulation. Therefore, extensive drug 1-drug 2 interaction and drug 1-drug 2-excipient studies are necessary to characterize prototype formulations. The additional number of variables in excipients selection and composition increases risk and also drives up development costs. Skilled formulators can create sophisticated experimental matrices and eliminate extraneous testing based on their experience, but the nature of risk dictates that it will increase with the number of test variables and possible outcomes regardless. Employing modular design in accordance with the present invention limits and mitigates this risk.

With monolithic dosage forms, commercial challenges are also encountered. During processing, the combination of multiple actives, especially when their physical characteristics are varied, e.g. large particle size vs. micronized drug particles, creates blends prone to segregation. Furthermore, disparate dose strengths, e.g. 500 mg vs. 2.5 mg, require extensive blend uniformity studies and process validation to demonstrate adequate control of the process. In spite of the cost and challenges monolithic dosage form development and manufacture present, their perceived simplicity makes them favored as a first step for most development efforts.

### Bilayered Tablets

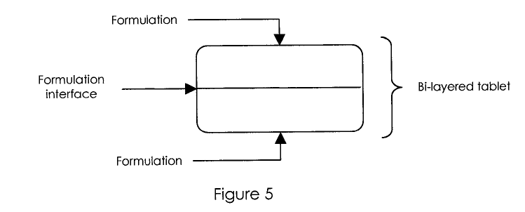

While bilayered tablets incorporate some elements of modular design at a lower level of granularity, the interface between the separate halves of the tablet still allows for drug formulation 1-drug formulation 2 interaction and drug 1-drug 2-excipient interactions (see FIG. 5). Therefore, even though each formulation is an independent module and processed separately until being merged during compression, the burden of drug and excipients compatibility testing is still required for each new combination envisioned.

### Capsule in Capsule or Tablet in Capsule:

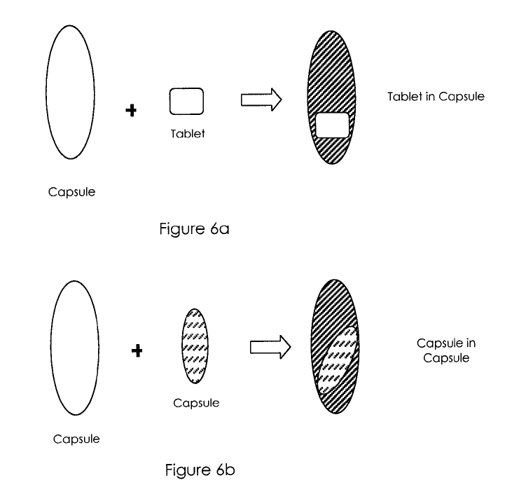

It is also possible to form fixed dose combinational dosage forms by placing a tablet or capsule containing one drug formulation within another capsule continuing a second formulation (See FIG. 6a, 6b). However, with such designs the performance of the interior dosage form will be affected by the exterior dosage, i.e. a sequential dissolution is unavoidable. While the performance may be desirable for certain applications, it represents a limitation to this dosage form from the standpoint of flexibility.

### Multi-Unit Systems:

In this formulation and processing approach, modularity is achieved by utilizing two or more formulations of coated particles. The level of modular granularity is similar to the bi-layered tablet because the independent formulation modules are merged in a single unit operation to yield the final fixed dose combination product. It differs from bi-layered tablets because the final coated particles preclude the interface between formulations and therefore can reduce and mitigate formulation development testing and risk.

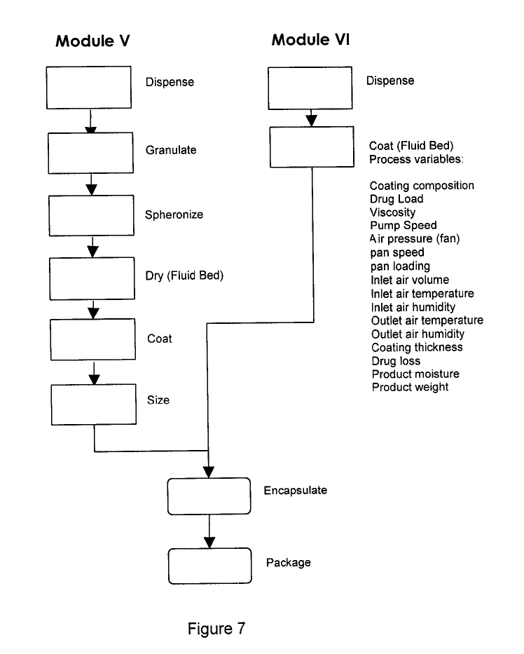

Multi unit systems are unique in that drug can be placed in either the core or the coating of each particle. Furthermore, with the flexibility of different coating material options, simultaneous or differential release profiles are possible. However, coating operations can add complexity through the myriad of processing variables that require characterization and control. In FIG. 7, Module V represents 6 unit operations necessary to blend formulation 1 with the drug in the core. Module VI represents how a nonpareil bead can be coated with active drug. In the box is a sampling of variables an operator might consider and control during processing.

As will be discussed below, the design and manufacture of dual chamber (bicameral) or barrier capsules comprising three-piece capsules in accordance with the present invention affords a high degree of modular granularity without restricting formulation options. By the very nature of capsules, formulated fills can include powders, granulations, pellets, beads (coated and uncoated), tablets or liquids. The barrier design of the three-piece capsule creates two separate compartments that avoids intermingling of the formulations, and isolates each formulation module without the need for complicated coating operations and eliminates drug-drug-excipient incompatibility issues between each formulation. These are clear advantages over existing fixed dose combination techniques.

Also, the core capsule and/or half-capsule walls may be selected to have a physical property such as thickness, composition, solubility and porosity whereby release of active pharmaceutical formulations contained therein into the alimentary canal may be controlled.

### EXAMPLES

The invention will now be illustrated in connection with the following working examples. As illustrated in FIGS. 2, 3 and 4, the process for filling a dual chamber or three piece capsule in accordance with the present invention involves two separate modules. The primary module (Module I, FIGS. 2 and 3; Module IV in FIG. 4) encapsulates a discrete formulation and creates a finished single entity product that can be warehoused or packaged and sold independently. It also can continue in the process immediately or after some storage to merge with the secondary module (Module II, FIG. 2 and 4; Module III in FIG. 3) to form a finished fixed dose combination product. Utilizing the modular approach, predefined and validated modules would not require process development, characterization through extensive testing and validation for each novel fixed dose combination. Only the new modules would require this level of testing. In this manner, development and manufacturing costs can be contained, delay to market time reduced, and risks can be minimized.

Equipment necessary to perform each unit operation for formulation of solid and liquid oral dosages is well-established in industry. Thus, it is a simple matter to modify an existing machine to merge the primary and secondary modules in the final encapsulation step in accordance with the present invention.

As discussed in our aforesaid parent patents and patent applications, there are many combinations of drugs that advantageously may be employed for treatment of co-morbid diseases, polypharmacy and/or reduce side effects of treatment. By way of example, eighty plus percent of diabetics reportedly are also hypertensive. Hyperlipidemia also is frequently concurrent with diabetes. Thus, an anti-diabetic agent conventionally used for treating diabetes such as a sulfonylurea, a meglitinide, a biguanide, an insulin sensitizer such as thiazolidinedione, or an alpha-glucosidase inhibitor may be combined with a drug useful for treating hypertension or hyperlipidemia. For example, a dose of sulfonylurea (e.g., Glipizide) can be combined in a single delivery system with a dose of a statin (e.g., Atorvastatin), a fibrate, a bile acid sequestrant (e.g., Cholestipol), a cholesterol absorption inhibitor or niacin. Likewise, a sulfonylurea can be combined with a bile acid sequestrant. Similarly, a drug for treating diabetes may be combined with an ACE inhibitor, an angiotension II antagonist, a calcium blocker, a beta-blocker, or a diuretic. An example is a combination of a biguanide (e.g., Metformin) coadministered with a calcium channel blocker (e.g., Amlodipine). Another example would be the combination of a meglitinide (e.g., Repaglinide) and an angiotension II antagonist (e.g., Losartan). Also, drug combinations may be selected based on the following criteria:
The possibility of a pharmacodynamic interaction. Drug combinations may be selected which exhibit affinity for the same receptors or may produce similar effects on physiologic function, related or not to their mechanism of action.
The possibility of a pharmacokinetic interaction. A pharmacokinetic interaction can manifest in several ways, some of which can be monitored in vivo and some of which cannot. One drug product may be selected based on its ability to alter the absorption or excretion of another product, change its distribution into one or more tissues, or change its pattern or rate of metabolism. Drugs may compete for serum protein binding, resulting in an increase in circulating free levels and tissue uptake of one drug.
The possibility of a toxicologic interaction (e.g., where the target organs for toxicity are similar for each drug). A possible lowering of a previously determined no-effect dose for one or both drug products and/or more severe toxicities in the affected organs should be considered, where applicable.
The margin of safety for each drug product. If one or more of the drugs has a narrow margin of safety (i.e., causes serious toxicity at exposures close to the predicted clinical exposure), then the possibility of drug interaction needs to be considered.
The possibility that the drugs compete for or alter the activity or endogenous levels of the same enzymes or other intracellular molecules should be considered (e.g., co-administration of two prooxidants could deplete endogenous levels of glutathione).
The possibility of a chemical interaction. One drug may chemically modify another drug (e.g., one drug may oxidize, methylate, or ethylate the other drug). This could result in new molecular entities with new toxicities. However, this effect can largely be avoided by providing for delayed release of one of the drugs.
The possibility that one drug may compromise the effectiveness of another drug.

Various embodiments of the invention will now be further described with reference to the following non-limiting examples:

(1) Combination #1: Enalapril maleate¹ and analogs and isomers thereof are ACE inhibitors used for the treatment of hypertension. This drug may be used with the following and analogs and isomers of beta adrenergic-blocking agents, methyldopa, nitrate, calcium blocking agents, Hydralazine⁶, Prazosin⁷ and Digoxin⁸ without clinically significant side effects. One or more of these agents may be packaged as above described with a drug for treatment of diabetes such as a sulfonylurea, a meglitimide, a biguanide, an insulin sensitizer or an alpha-glucosidase inhibitor.

(2) Combination #2: A hypoglycermic agent such as Metformin HCl² and analogs and isomers thereof may be packaged as above described with an angiotensin converting enzyme inhibitor (ACE inhibitor).

(3) Combination #3: A diabetes drug as above described in Combination #1 or #2 may be packaged as above described with an angiotensin II receptor antagonist such as Losartan potassium³ and/or Valsartan⁴.

(4) Combination #4: A diabetes drug as above described may be packaged as above described with a Beta Adrenergic Blocking Agent such as Bioprolol fumarate⁵ or Metoprolol succinate⁶.

(5) Combination #5: A diabetes drug as above described may be packaged as described in Combinations #1 or #2 may be packaged with a Calcium Channel Blocking Agent such as Amlodipine⁷ or Nifedipine⁸.

(6) Combination #6: A diabetes drug as above described may be packaged with a Periferal Adrenergic Blocking Agent such as Prazosin hydrochloride⁹.

(7) Combination #7: A diabetes drug as above described may be packaged with an Adrenergic central stimulant such as Methyldopa¹⁰ or Clonidine¹¹.

(8) Combination #8: A biguanide such as Metformin¹⁴ may be packaged as above described with a sulfonylurea such as Glipizide¹⁵.

(9) Combination #9: A biguanide such as Metformin¹⁴ may be packaged as above described with a thiazolidinedione such as rosiglitazone maleate¹⁶.

(10) Combination #10: A biguanide such as Metfonnin¹⁴ may be packaged as above described with an alpha glucosidase inhibitor such as Cerivastatin¹⁷.

(11) Combination #11: A short acting oral insulin may be packaged as above described with sustained release oral insulin.

The drug delivery system of the present invention also allows three drug combinations such as diabetes drugs and ACE Inhibitors combined with Beta Blockers, methyldopa nitrates, calcium channel blockers, Hydralazine¹², Prazosin¹³, Digoxin¹⁴ as well as multiple combinations of drugs.

(12) Combination #12: A diabetes drug may be packaged with an ACE Inhibitor and a Beta Blocker.

(13) Combination #13: A diabetes drug such as described in Combinations #1 or #2 may be packaged with a HMG-CoA reductase inhibitor such as Simvastatin³⁵, Atorvastatin³⁶, or Pravastatin³⁷, and with a bile acid sequestrant such as Colestipol hydrohloride³⁸.

(14) Combination #14: A diabetes drug such as described in Combinations #1 or #2 may be packaged with a HMG-CoA reductase inhibitor and with a niacin compound.

(15) Combination #15: A diabetes drug such as described in Combinations #1 or #2 may be packaged with a HMG-CoA reductase inhibitor or Combination #14, and with a hypolipidemia agent such as Gemfibrozil³⁹.

While the above embodiments of the invention has been described with particular drug combinations segregated from one another, it will be understood that some of the above-listed drug combinations also may be blended and packaged in a single tablet, capsule or caplet if a more traditional manufacturing approach is desirable.

Other embodiments of the present invention are directed towards combinations of at least one active pharmaceutical ingredient and at least one substance which can be an active pharmaceutical ingredient or non- pharmaceutical ingredient and which is mitigating the negative effects of said first active pharmaceutical ingredient, or promoting/enhancing action of said first active pharmaceutical ingredient, or is promoting general health and well-being of the patient taking said first active pharmaceutical ingredient. The following non-limiting examples are illustrating this aspect of the embodiments of the present invention:

### Example 16

A combination of first active pharmaceutical ingredient which may cause a side effect with a second active pharmaceutical ingredient medication mitigating side effect of the first active pharmaceutical ingredient are combined in a single delivery package. Examples include first active pharmaceutical ingredient with side effect causing, constipation, nausea, gas/bloating, heartburn, pain or cramps; and a second active pharmaceutical ingredient, mitigating the above side effect of the first ingredient, e.g. correspondingly laxative medication, nausea treatment medication, anti-gas and anti-bloating medication, anti-acid medication, pain reliever & muscle relaxant medication. More specific example may include pain medication causing constipation and nausea, e.g. oral narcotic with the second ingredient containing stool softener and anti-nausea components.

### Example 17

In another embodiment of the present invention, a first active pharmaceutical ingredient is combined with a second active pharmaceutical ingredient which controls and stops the action of the first ingredient after the time necessary for the action of the first ingredient. As an example, a combination of anti-cancer drug such as Methotrexate with immediate release, and the “quencher” substance, such as L-leukovorin, with delayed release, can be advantageously delivered within the combination medication delivery system.

### Example 18

In another embodiment of the present invention, a first active pharmaceutical ingredient is combined with a second active pharmaceutical ingredient or a substance which optimizes the pH in the immediate vicinity of the first active pharmaceutical ingredient for facilitating dissolution, and/or absorption of the first active pharmaceutical ingredient. Additionally, control and/or neutralization of the stomach acid to slow down first active pharmaceutical ingredient breakdown can be affected thus improving the bioavailability of the first active pharmaceutical ingredient. Non-limiting examples of pH controlling substances include pH buffering compounds known in the art.

### Example 19

In another embodiment of the present invention, a first active pharmaceutical ingredient which is fat soluble is combined with a second active pharmaceutical ingredient or a substance containing oil for better drug solubility and absorption.

### Example 20

In another embodiment of the present invention, a first active pharmaceutical ingredient is combined with an enzyme wherein said enzyme facilitates active pharmaceutical ingredient absorption and/or bio-availability or mitigates side effects.

### Example 21

In another embodiment of the present invention, a first active pharmaceutical ingredient is combined with a nutraceutical or a vitamin. Non-limiting examples include combination of (i) Nexium (esomeprazole) which changes the pH in the stomach and thus prevents absorption of B12 vitamin which can only happen at low pH, with B-group vitamins and (ii) Anti-viral active pharmaceutical ingredients with vitamin C or multivitamin supplements.

### Example 22

In another embodiment of the present invention, a first active pharmaceutical ingredient is combined with a surfactant which facilitates absorption or vice versa, inhibits absorption in the certain part of the alimentary canal.

### Example 23

In another embodiment of the present invention, a first active pharmaceutical ingredient is combined with a sleep aid.

Another embodiment of the present invention is directed towards combinations of at least two active pharmaceutical ingredients within the same class of pharmaceuticals treating or preventing the same symptoms or same disease (polypharmacy), such as infectious disease, metabolic disorders, cardiovascular disease, pain, cancer, transplant-related treatment, gastrointestinal disorders, respiratory diseases, autoimmune diseases, vaccines, etc. The following non-limiting examples are illustrating this embodiment of the present invention:

### Example 24

Combination of anti-infective active pharmaceutical ingredients, with examples including at least two antibiotics combined, resulting in a broad spectrum anti-bacterial action. Another example includes a combination of anti-viral and anti-bacterial pharmaceutical ingredients resulting in a treatment of an infection with unknown pathogen as well as treatment of bacterial infections often following viral infections. Yet another example includes a combination of at least two active pharmaceutical ingredients which are treating cancer or managing the symptoms of cancer, for example topoisomerase inhibitor drug and anti-cancer monoclonal antibody drug. Another example includes a combination of antibiotic with antibiotic potentiators. Potentiators confer increased activity to pharmaceutical agents, such as, for instance, antibiotics. Although potentiators may lack themselves any antibacterial activity, in combination with antibiotics, such as for example, erythromycin, chloramphenicol, tetracycline, linezolid, clindamycin or rifampin, potentiators promote and significantly increase the activity of the pharmaceutical agent, in this example, antibiotic.

### Example 25

In another embodiment of the present invention, the same active pharmaceutical ingredient is combined in at least two formulations, including a fast release or fast action and a slow release or long term action formulation. The slow release or long term action can be achieved by differential release capsule components design, as discussed above, or by formulation of the drug, excipients and tablet forming means, and other means available to these skilled in the art, with beneficial effects including better treatment or relief of symptoms and potential for the decrease of the overall medication intake. Specific non-limiting examples include: nitroglycerin, with fast acting/fast dissolving formulation providing for a fast action for acute treatment with a slow release formulation for maintenance; antibiotic with fast action / fast dissolution formulation for immediate increase of the concentration in blood plus slow release; pain medication, with a fast acting formulation for immediate pain relief help combined with a slow release pain maintenance medication; sleep aid with a fast dissolving or fast acting formulation for immediate effect combined with a delayed release for maintenance throughout the night, with specific non-limiting example including Ambien.

### Example 26

In another embodiment of the present invention, at least two anti-cholesterol pharmaceutical ingredients such as statins of different types are combined in the combination medication delivery system. Since effects of statins are highly individual, a combination medication is advantageous.

### Example 27

In another embodiment of the present invention, a broad spectrum anti-hypertensive combination comprises two or more hypertension-reducing drugs in the combination medication delivery system, including medications of the same type, such as beta-blockers or diuretics, or medications of different types or classes, such as beta-blocker and diuretic.

Various other changes may be made without departing from the spirit and scope of the invention. For example, the above-described capsules may be used with various drug combinations as described in U.S. Pat. Nos. 6,428,809 and 6,702,783, and the drug combinations described in co-pending application Ser. Nos. 10/756,124 and 10/479,438. Still other drug combinations, which term may also include vitamins, dietary supplements, minerals and nutraceuticals, which may be used with the above-described capsules or with the combination capsules, tablets or caplets described in our earlier patents and pending applications, include combination drug therapies for treating infectious disease, e.g., AIDS, TB and malaria, and for pain management, e.g., nonsteroidal anti-inflammatory drugs/proton pump inhibitors (NSAIDS/PPI). These include, by way of example, and not limitation:

### Example 28

In another embodiment of the present invention, at least two anti-malaria drugs are combined in the combination medication delivery system. Specific Examples of potential drug combinations include, Artesunate and Mefloquine; Artemether and Lumefantrine; Chloroquine and Paracetamol. More generally, a combination of at least two of the following representative anti-malaria drugs in the combination medication delivery system are exemplified: Artemether; Lumefantrine; Artensunate; Amodiaquine HCl; Atovaquone-proguanil; Quinine Sulfate; Chloroquine Sulfate; Hydroxychloroquine Sulfate; Doxycycline; Mefloquine; Primaquine; Sulfadoxine; Pyrimethamine; Paracetamol.

### Example 29

In another embodiment of the present invention, at least two HIV treatment medications are combined in the combination medication delivery system. Specific Examples of potential drug combinations include, at least two of the nucleoside reverse transcriptase inhibitor (NRTI) medications, including e.g. Abacavir; lamivudine; Didanosine; Emtricitabine; Stavudine; Tenofovir. Another example includes combining a non-nucleoside reverse transcriptase inhibitor (NNRTI) and a nucleoside reverse transcriptase inhibitor (NRTI) e.g. Nevirapine (NNRTI) and didanozine (NRTI); Efavirenz (NNRTI) and abacavir sulfate (NRTI). Yet another example includes combining two NRTI's and one NNRTI e.g. Abacavir and lamivudine and efavirenz or Abacavir and lamivudine and nevirapine. Still another Example includes combining at least two 2 NRTI's and a PPI: Abacavir and lamivudine and lopinavir/ritonavir. Still another example includes a combination of at least two of the anti-HIV drugs selected from the group comprising: abacavir sulfate; didanozine; stavudine; tenofovir; disoproxil; ftimarate; zidovudine; lamivudine; emtricitabine; lopinavir/ritonavir; nevirapine; efavirenz; nelfinavir. Still other combinations include combination of AZT and 3TC; combination of abacavir and AZT and 3TC; a combination of lopinavir and ritonavir; combinations of ABC and 3TC; and combination of emtricitabine and tenofovir.

### Example 30

In another embodiment of the present invention, at least two of Tuberculosis treatment medications are combined in the combination medication delivery system. Specific Examples of potential combinations include at least two of the following medications: Isoniazid; Rifampicin; Pyrazinamide; Ethambutol HCl; Streptomycin; Capreomycin; Cycloserine; Protionamide; Macrolides; Fluoroquinolones; p-Salicylic acid.

### Example 31

In another embodiment of the present invention, at least two of the pain treatment medications are combined in the combination medication delivery system. Specific Examples of potential combinations include at least two of the following medications: Aspirin; Carbex; Codeine; Luvox; Marplan; Nardil; Neurotin; OxyContin; Parnate; Topamax; Tylenol/Acetaminophen; Vicodin; Xyrem; Zarontin; Zoloft; Zomig.

### Example 32

Another embodiment of the present invention is a combination of aspirin or acetylsalicylic acid combined in the combination medication delivery system with a active ingredient mitigating side effects of aspirin, such as effects related to the acidity of aspirin. Specific Examples of potential combinations include buffering compounds and anti-acid compounds in combination with aspirin.

### Example 33

Another embodiment of the present invention is a combination therapy for treatment of lupus nephritis. Specific example includes combination of methylprednisolone and cyclophosphamide.

Still other changes are possible. For example, a pre-formed tablet, capsule or caplet containing one pharmaceutical ingredient may be obtained from the manufacturer. Then, a compounding pharmacist may load that pre-formed tablet within one compartment of the three piece capsule, and load the second pharmaceutical ingredient into the second compartment. This permits a compounding pharmacist to produce custom drug combination packages.

Various other changes may be possible without departing from the spirit and scope of the invention. For example, the core may comprise a capsule containing a liquid or gel. Still other changes are possible.

1. A pharmaceutical delivery package comprising fixed unit dose quantities of two or more different pharmaceutical formulations (a) combined in a single delivery package, and (b) segregated from one another within said package wherein said package comprises a core capsule containing a first pharmaceutical formulation surrounded at least in part by a half-capsule containing a second pharmaceutical formulation.

2. A pharmaceutical delivery package according to claim 1, wherein the core capsule is formed from gelatin, a starch or a cellulose material.

3. A pharmaceutical delivery package according to claim 2, wherein the cellulose material comprises hydroxypropylmcthylcelluose.

4. A pharmaceutical delivery package according to claim 1, wherein said core capsule and said half capsule are joined together by snap or press fitting.

5. A pharmaceutical delivery package according to claim 1, wherein at least one of the two or more different pharmaceutical formulations is in a powder, pellet or bead form.

6. A pharmaceutical delivery package according to claim 1, wherein at least one of said first and said second pharmaceutical formulations is in a semi-liquid or gel form.

7. A pharmaceutical delivery package according to claim 1, wherein at least one of said first and said second pharmaceutical formulations is in a pre-formed dose form.

8. A pharmaceutical delivery package according to claim 1, wherein the core capsule and the half-capsule are bonded to one another.

9. A pharmaceutical delivery package according to claim 1, wherein the core capsule and the half-capsule are joined together by mating rings, a locking groove and ring, or a locking band.

10. A pharmaceutical delivery package according to claim 1, wherein the core capsule and the half-capsule are joined together by a set-in-place liquid.

11. A pharmaceutical delivery package according to claim 1, wherein the core capsule and/or the half-capsule walls have a physical property selected from thickness, composition, solubility and porosity whereby release of active pharmaceutical formulations contained therein into the alimentary canal may be controlled.

12. A pharmaceutical delivery package according to claim 11, wherein the core capsule and/or the half-capsule walls are acid resistant, and are permeable or soluble in a neutral to alkaline environment.

13. A pharmaceutical delivery package according to claim 1, wherein the core 11 capsule contains a liquid or gel formulation.

14. A pharmaceutical delivery package according to claim 1, wherein one of the pharmaceutical formulations is selected from the group consisting of a vitamin, a dietary supplement, a mineral and a nutraceutical.

15. A pharmaceutical delivery package according to claim 1, comprising combinations of pharmaceutical formulations selected from the group consisting of an anti-diabetic agent and an anti-hypertensive agent; an anti-diabetic agent and anti-hyperlipidemia agent, wherein the anti-diabetic agent preferably is selected from the group consisting of a sulfonylurea, a meglitinide, a biguanide, an insulin sensitizer and an alpha-glucosidase inhibitor, and the anti-hypertensive agent preferably is selected from the group consisting of an ACE inhibitor, an angiotension II antagonist, a calcium blocker, a beta-blocker and a diuretic, or wherein the anti-diabetic agent preferably is selected from the group consisting of a sulfonylurea, a meglitinide, a biguanide, an insulin sensitizer and an alpha-glucosidase inhibitor, and the anti-hyperlipidemia agent preferably is selected from the group consisting of a statin, a fibrate, a bile acid sequestrant, a cholesterol absorption inhibitor and niacin.

16. A pharmaceutical delivery package according to claim 1, wherein one of the pharmaceutical formulations is selected from an ingredient which mitigates a side effect of the other pharmaceutical formulation; or which acts as a time control quencher for the other pharmaceutical formulation; or which facilitates dissolution and/or absorption of the other pharmaceutical formulation, e.g., through pH control; or, which is fat soluble and the other pharmaceutical formulation contains a fat or oil; or which contains an enzyme for facilitating absorption and/or bio-availability of the other pharmaceutical formulation, or mitigating side effects of the other pharmaceutical formulation; or, which includes a surfactant which facilitates absorption or inhibits absorption in a selected part of the alimentary canal; or which comprises a sleep aid.

17. A pharmaceutical delivery package according to claim 1, wherein the first and the second pharmaceutical formulations are effective for treating the same symptom or disease; or the first and the second pharmaceutical formulations are both antibiotics; or one of the pharmaceutical formulations is an anti-viral agent, and the other pharmaceutical formulation is an anti-bacterial agent; or one of the pharmaceutical formulations is an antibiotic, and the other pharmaceutical formulation is an antibiotic potentiator; or one of the pharmaceutical formulations comprises an NRTI and the other pharmaceutical formulation comprises an NNRTI; or one of the pharmaceutical formulations comprises a PPI, and the other pharmaceutical formulation comprises an NNRTI or one of the pharmaceutical formulations comprises an NSAID, and the other pharmaceutical formulation comprises a PPI.

18. A pharmaceutical delivery package according to claim 1, wherein the pharmaceutical formulations comprise agents for treating infectious disease or pain.

19. A pharmaceutical delivery package according to claim 18, wherein the infectious disease comprises HIV/AIDS, TB or malaria.

20. A pharmaceutical delivery package according to claim 1, comprising two or more pharmaceutical formulations selected from the group consisting of Enalapril maleate and analogs and isomers thereof and analogs and isomers of beta adrenergic-blocking agents, methyldopa, nitrate, calcium blocking agents, Hydralazine, Prazosin and Digoxin; a hypoglycermic agent such as Metformin HCl and analogs and isomers thereof and an angiotensin converting enzyme inhibitor (ACE inhibitor); a diabetes drug and an angiotensin II receptor antagonist such as Losartan potassium and/or Valsartan; a diabetes drug and a Beta Adrenergic Blocking Agent such as Bioprolol fumarate or Metoprolol succinate; a diabetes drug and a Calcium Channel Blocking Agent such as Amlodipine or Nifedipine; a diabetes drug and a Periferal Adrenergic Blocking Agent such as Prazosin hydrochloride; a diabetes drug and a Adrenergic central stimulant such as Methyldopa or Clonidine; a biguanide such as Metformin and a sulfonylurea such as Glipizide; a biguanide such as Metformin and a thiazolidinedione such as rosiglitazone maleate; a biguanide such as Metformin and an alpha glucosidase inhibitor such as Cerivastatin; a short acting oral insulin and a sustained release oral insulin; a diabetes drug and an ACE Inhibitor combined with a Beta Blocker, a methyldopa nitrate, a calcium channel blocker, Hydralazine, Prazosin, or Digoxin; a diabetes drug and an ACE Inhibitor and a Beta Blocker; a diabetes drug and a HMG-CoA reductase inhibitor such as Simvastatin, Atorvastatin, or Pravastatin, and with a bile acid sequestrant such as Colestipol hydrohloride; a diabetes drug and a HMG-CoA reductase inhibitor and a niacin compound; a diabetes drug and a HMG-CoA reductase inhibitor or Combination, and with a hypolipidemia agent such as Gemfibrozil; a pharmaceutical formulation with side effect causing, constipation, nausea, gas/bloating, heartburn, pain or cramp and a second pharmaceutical formulation, mitigating the above side effect of the first formulation, e.g. correspondingly laxative medication, nausea treatment medication, anti-gas and anti-bloating medication, anti-acid medication, pain reliever & muscle relaxant medication; a pain medication causing constipation and nausea, oral narcotic and the second formulation containing a stool softener and/or an anti-nausea components; an anti-cancer drug such as Methotrexate with immediate release, and a “quencher” substance, such as L-leukovorin, with delayed release; a first pharmaceutical formulation and a second pharmaceutical formulation or a substance which optimizes or controls pH such as a buffer for facilitating dissolution, and/or absorption of the first active pharmaceutical formulation; a first pharmaceutical formulation which is fat soluble and a second pharmaceutical formulation or a substance containing oil; a first pharmaceutical formulation and an enzyme wherein said enzyme facilitates active pharmaceutical formulation absorption and/or bio-availability or mitigates side effects; a first pharmaceutical formulation and a nutraceutical or a vitamin, such as Nexium (esomeprazole) and B-group vitamins, and Anti-viral active pharmaceutical formulations and vitamin C or multivitamin supplements; a pharmaceutical formulation and a surfactant which facilitates absorption or vice versa, inhibits absorption in the certain part of the alimentary canal; a pharmaceutical formulation and a sleep aid; a first and second formulation within the same class of pharmaceuticals for treating or preventing the same symptoms or same disease (polypharmacy), such as infectious disease, metabolic disorders, cardiovascular disease, pain, cancer, transplant-related treatment, gastrointestinal disorders, respiratory diseases, autoimmune diseases and vaccines; an anti-infective active pharmaceutical formulation comprising first and second antibiotics; an anti-viral and an anti-bacterial pharmaceutical formulation; a pharmaceutical formulation, for treating cancer and managing symptoms of cancer, for example topoisomerase inhibitor drug, and an anti-cancer monoclonal antibody drug, an antibiotic and an antibiotic potentiator; a fast release or fast action and slow release or long term action formulations of the same pharmaceutical, such as nitroglycerin, with fast acting/fast dissolving formulation providing for a fast action for acute treatment with a slow release formulation for maintenance; an antibiotic with fast action/fast dissolution formulation for immediate increase of the concentration in blood plus slow release; pain medication, with a fast acting formulation for immediate pain relief help combined with a slow release pain maintenance medication; a sleep aid with a fast dissolving or fast acting formulation for immediate effect combined with a delayed release for maintenance throughout the night, such as Ambien; two anti-cholesterol pharmaceutical formulations such as statins of different types combined in the combination medication delivery system; a broad spectrum anti-hypertensive combination comprising two or more hypertension-reducing drugs, including medications of the same type, such as beta-blockers or diuretics, or medications of different types or classes, such as beta-blocker and diuretic; two or more anti-malaria drugs such as Artesunate and Mefloquine; Artemether and Lumefantrine; Chloroquine and Paracetamol; and at least two of the following: Artemether; Lumefantrine; Artensunate; Amodiaquine HCl; Atovaquone-proguanil; Quinine Sulfate; Chloroquine Sulfate; Hydroxychloroquine Sulfate; Doxycycline; Mefloquine; Primaquine; Sulfadoxine; Pyrimethamine; Paracetamol; at least two nucleoside reverse transcriptase inhibitor (NRTI) medications, including e.g. Abacavir; lamivudine; Didanosine; Emtricitabine; Stavudine; Tenofovir, a non-nucleoside reverse transcriptase inhibitor (NNRTI) and a nucleoside reverse transcriptase inhibitor (NRTI) e.g. Nevirapine (NNRTI) and didanozine (NRTI); Efavirenz (NNRTI) and abacavir sulfate (NRTI); two NRTI's and one NNRTI, e.g. Abacavir and lamivudine and efavirenz or Abacavir and lamivudine and nevirapine; at least two 2 NRTI's and a PPI such as Abacavir and lamivudine and lopinavir/ritonavir; at least two anti-HIV drug formulations selected from the group consisting of: abacavir sulfate; didanozine; stavudine; tenofovir; disoproxil; fumarate; zidovudine; lamivudine; emtricitabine; lopinavir/ritonavir; nevirapine; efavirenz and nelfinavir; a combination of AZT and 3TC; a combination of abacavir and AZT and 3TC; a combination of lopinavir and ritonavir; combinations of ABC and 3TC; a combination of emtricitabine and tenofovir; at least two of Tuberculosis treatment medications selected from: Isoniazid; Rifampicin; Pyrazinamide; Ethambutol HCl; Streptomycin; Capreomycin; Cycloserine; Protionamide; Macrolides; Fluoroquinolones; and p-Salicylic acid; at least two of the pain treatment medications selected from: Aspirin; Carbex; Codeine; Luvox; Marplan; Nardil; Neurotin; OxyContin; Parnate; Topamax; Tylenol/Acetaminophen; Vicodin; Xyrem; Zarontin; Zoloft and Zomig; a pH buffering compound and/or an anti-acid compound in combination with aspirin; and a combination therapy for treatment of lupus nephritis, such as methylprednisolone and cyclophosphamide.

21. A pharmaceutical delivery package as claimed in claim 1 comprising fixed unit dose quantities of two or more different active pharmaceutical formulations (a) combined in a single delivery package, and (b) segregated from one another within said package, characterized by one or more of the following features:
(a) wherein one of the pharmaceutical formulations is selected from the group consisting of a vitamin, a dietary supplement, a mineral and a nutraceutical;
(b) comprising combinations of pharmaceutical formulations selected from the group consisting of an anti-diabetic agent and an anti-hypertensive agent; an anti-diabetic agent and anti-hyperlipidemia agent, wherein the anti-diabetic agent preferably is selected from the group consisting of a sulfonylurea, a meglitinide, a biguanide, an insulin sensitizer and an alpha-glucosidase inhibitor, and the anti-hypertensive agent preferably is selected from the group consisting of an ACE inhibitor, an angiotension II antagonist, a calcium blocker, a beta-blocker and a diuretic, or wherein the anti-diabetic agent preferably is selected from the group consisting of a sulfonylurea, a meglitinide, a biguanide, an insulin sensitizer and an alpha-glucosidase inhibitor, and the anti-hyperlipidemia agent preferably is selected from the group consisting of a statin, a fibrate, a bile acid sequestrant, a cholesterol absorption inhibitor and niacin;
(c) wherein one of the pharmaceutical formulations is selected from an ingredient which mitigates a side effect of the other pharmaceutical formulation; or which acts as a time control quencher for the other pharmaceutical formulation; or which facilitates dissolution and/or absorption of the other pharmaceutical formulation, e.g., through pH control; or, which is fat soluble and the other pharmaceutical formulation contains a fat or oil; or which contains an enzyme for facilitating absorption and/or bio-availability of the other pharmaceutical formulation, or mitigating side effects of the other pharmaceutical formulation; or, which includes a surfactant which facilitates absorption or inhibits absorption in a selected part of the alimentary canal; or which comprises a sleep aid;
(d) wherein the first and the second pharmaceutical formulations are effective for treating the same symptom or disease; or the first and the second pharmaceutical formulations are both antibiotics; or one of the pharmaceutical formulations is an anti-viral agent, and the other pharmaceutical formulation is an anti-bacterial agent; or one of the pharmaceutical formulations is an antibiotic, and the other pharmaceutical formulation is an antibiotic potentiator; or one of the pharmaceutical formulations comprises an NRTI and the other pharmaceutical formulation comprises an NNRTI; or one of the pharmaceutical formulations comprises a PPI, and the other pharmaceutical formulation comprises an NNRTI or one of the pharmaceutical formulations comprises an NSAID, and the other pharmaceutical formulation comprises a PPI;
(e) wherein the pharmaceutical formulations comprise agents for treating infectious disease or pain;
(f) wherein the infectious disease comprises HIV/AIDS, TB or malaria; and
(g) comprising two or more pharmaceutical formulations selected from the group consisting of Enalapril maleate and analogs and isomers thereof and analogs and isomers of beta adrenergic-blocking agents, methyldopa, nitrate, calcium blocking agents, Hydralazine, Prazosin and Digoxin; a hypoglycermic agent such as Metformin HCl and analogs and isomers thereof and an angiotensin converting enzyme inhibitor (ACE inhibitor); a diabetes drug and an angiotensin II receptor antagonist such as Losartan potassium and/or Valsartan; a diabetes drug and a Beta Adrenergic Blocking Agent such as Bioprolol fumarate or Metoprolol succinate; a diabetes drug and a Calcium Channel Blocking Agent such as Amlodipine or Nifedipine; a diabetes drug and a Periferal Adrenergic Blocking Agent such as Prazosin hydrochloride; a diabetes drug and a Adrenergic central stimulant such as Methyldopa or Clonidine; a biguanide such as Metformin and a sulfonylurea such as Glipizide; a biguanide such as Metformin and a thiazolidinedione such as rosiglitazone maleate; a biguanide such as Metfornin and an alpha glucosidase inhibitor such as Cerivastatin; a short acting oral insulin and a sustained release oral insulin; a diabetes drug and an ACE Inhibitor combined with a Beta Blocker, a methyldopa nitrate, a calcium channel blocker, Hydralazine, Prazosin, or Digoxin; a diabetes drug and an ACE Inhibitor and a Beta Blocker; a diabetes drug and a HMG-CoA reductase inhibitor such as Simvastatin, Atorvastatin, or Pravastatin, and with a bile acid sequestrant such as Colestipol hydrohloride; a diabetes drug and a HMG-CoA reductase inhibitor and a niacin compound; a diabetes drug and a HMG-CoA reductase inhibitor or Combination, and with a hypolipidemia agent such as Gemfibrozil; a pharmaceutical formulation with side effect causing, constipation, nausea, gas/bloating, heartburn, pain or cramp and a second pharmaceutical formulation, mitigating the above side effect of the first ingredient, e.g. correspondingly laxative medication, nausea treatment medication, anti-gas and anti-bloating medication, anti-acid medication, pain reliever & muscle relaxant medication; a pain medication causing constipation and nausea, oral narcotic and the second formulation containing a stool softener and/or an anti-nausea components; an anti-cancer drug such as Methotrexate with immediate release, and a “quencher” substance, such as L-leukovorin, with delayed release; a first pharmaceutical formulation and a second pharmaceutical formulation or a substance which optimizes or controls pH such as a buffer for facilitating dissolution, and/or absorption of the first active pharmaceutical formulation; a first pharmaceutical formulation which is fat soluble and a second pharmaceutical formulation or a substance containing oil; a first pharmaceutical formulation and an enzyme wherein said enzyme facilitates active pharmaceutical formulation absorption and/or bio-availability or mitigates side effects; a first pharmaceutical formulation and a nutraceutical or a vitamin, such as Nexium (esomeprazole) and B-group vitamins, and Anti-viral active pharmaceutical formulations and vitamin C or multivitamin supplements; a pharmaceutical formulation and a surfactant which facilitates absorption or vice versa, inhibits absorption in the certain part of the alimentary canal; a pharmaceutical formulation and a sleep aid; a first and second formulation within the same class of pharmaceuticals for treating or preventing the same symptoms or same disease (polypharmacy), such as infectious disease, metabolic disorders, cardiovascular disease, pain, cancer, transplant-related treatment, gastrointestinal disorders, respiratory diseases, autoimmune diseases and vaccines; anti-infective active pharmaceutical formulation comprising first and second antibiotics; an anti-viral and an anti-bacterial pharmaceutical formulation; a pharmaceutical formulation, for treating cancer and managing symptoms of cancer, for example topoisomerase inhibitor drug, and an anti-cancer monoclonal antibody drug, an antibiotic and an antibiotic potentiator; a fast release or fast action and slow release or long term action formulations of the same pharmaceutical, such as nitroglycerin, with fast acting/fast dissolving formulation providing for a fast action for acute treatment with a slow release formulation for maintenance; antibiotic with fast action/fast dissolution formulation for immediate increase of the concentration in blood plus slow release; pain medication, with a fast acting formulation for immediate pain relief help combined with a slow release pain maintenance medication; sleep aid with a fast dissolving or fast acting formulation for immediate effect combined with a delayed release for maintenance throughout the night, such as Ambien; two anti-cholesterol pharmaceutical formulations such as statins of different types combined in the combination medication delivery system; a broad spectrum anti-hypertensive combination comprising two or more hypertension-reducing drugs, including medications of the same type, such as beta-blockers or diuretics, or medications of different types or classes, such as beta-blocker and diuretic; two or more anti-malaria drugs such as Artesunate and Mefloquine; Artemether and Lumefantrine; Chloroquine and Paracetamol; and at least two of the following: Artemether; Lumefantrine; Artensunate; Amodiaquine HCl; Atovaquone-proguanil; Quinine Sulfate; Chloroquine Sulfate; Hydroxychloroquine Sulfate; Doxycycline; Mefloquine; Primaquine; Sulfadoxine; Pyrimethamine; Paracetamol; at least two nucleoside reverse transcriptase inhibitor (NRTI) medications, including e.g. Abacavir; lamivudine; Didanosine; Emtricitabine; Stavudine; Tenofovir, a non-nucleoside reverse transcriptase inhibitor (NNRTI) and a nucleoside reverse transcriptase inhibitor (NRTI) e.g. Nevirapine (NNRTI) and didanozine (NRTI); Efavirenz (NNRTI) and abacavir sulfate (NRTI); two NRTI's and one NNRTI, e.g. Abacavir and lamivudine and efavirenz or Abacavir and lamivudine and nevirapine; at least two 2 NRTI's and a PPI such as Abacavir and lamivudine and lopinavir/ritonavir; at least two anti-HIV drug formulations selected from the group consisting of: abacavir sulfate; didanozine; stavudine; tenofovir; disoproxil; fumarate; zidovudine; lamivudine; emtricitabine; lopinavir/ritonavir; nevirapine; efavirenz and nelfinavir; a combination of AZT and 3TC; a combination of abacavir and AZT and 3TC; a combination of lopinavir and ritonavir; combinations of ABC and 3TC; a combination of emtricitabine and tenofovir; at least two of Tuberculosis treatment medications selected from: Isoniazid; Rifampicin; Pyrazinamide; Ethambutol HCl; Streptomycin; Capreomycin; Cycloserine; Protionamide; Macrolides; Fluoroquinolones; and p-Salicylic acid; at least two of the pain treatment medications selected from: Aspirin; Carbex; Codeine; Luvox; Marplan; Nardil; Neurotin; OxyContin; Parnate; Topamax; Tylenol/Acetaminophen; Vicodin; Xyrem; Zarontin; Zoloft and Zomig; a pH buffering compound and/or an anti-acid compound in combination with aspirin; and a combination therapy for treatment of lupus nephritis, such as methylprednisolone and cyclophosphamide.

22. A process for packaging of two or more different pharmaceutical formulations in a single delivery package, comprising providing a dose of a first pharmaceutical formulation, in a primary process module, and combining the dose of the first pharmaceutical formulation with a dose of a second pharmaceutical formulation from a secondary process module.

23. A pharmaceutical delivery package as claimed in claim 1, comprising fixed unit dose quantities of two or more different pharmaceutical formulations (a) combined in a single delivery package, and (b) segregated from one another within said package, formed by the process of claim 22.

24. A modular pharmaceutical delivery package comprising a tablet-in-a-capsule, formed by the process of claim 22.

25. A modular pharmaceutical delivery package comprising a three piece capsule or a capsule-in-a-capsule, formed by the process of claim 22.

26. The process of claim 25, wherein the primary process module comprises a pharmaceutical formulation encapsulated in a two-piece capsule, and the secondary process module comprises a second pharmaceutical formulation loaded in a half-capsule and merged with the primary process module.

27. The process of claim 25, wherein the first pharmaceutical ingredient is formed as a segregated tablet or capsule which is then loaded, together with the second pharmaceutical formulation, in a capsule.

28. The process of claim 26, wherein the first pharmaceutical formulation is encapsulated or delivered from bulk to said two-piece capsule, and the second pharmaceutical formulation is delivered from bulk to said second half capsule which is joined to the two-piece capsule.

29. The process of claim 25, wherein the first and/or the second pharmaceutical formulations are prepared in separate process streams.

30. The process of claim 29, wherein at least one of the process streams includes one or more of the steps of mixing, blending, screening, granulating, wetting and drying, milling, coating and/or compressing.

31. A process for packaging two or more different pharmaceutical formulations in a single delivery package, comprising encapsulating a unit dose quantity of said first pharmaceutical formulation in a capsule, loading a dose quantity of a second pharmaceutical formulation in a half capsule, and, joining the half capsule to the capsule.

32. A process for packaging two or more different pharmaceutical formulations in a single delivery package, comprising providing a first dose quantity of a first pharmaceutical formulation, encapsulated in a first capsule, and supplying a dose quantity of a second pharmaceutical formulation in a second capsule, and joining the first capsule to the second capsule.

33. A process for manufacturing a pharmaceutical delivery package comprising unit dose quantities of two or more different pharmaceutical formulations combined in a single delivery package which comprises providing said two or more pharmaceutical formulations in separate process modules; loading one of the pharmaceutical formulations in an isolated compartment in a capsule; adding the second pharmaceutical formulation to the capsule; and
sealing the capsule to create a single delivery package

34. The process as claimed in claim 33, wherein the capsule comprises a three-piece capsule.

35. The process as claimed in claim 34, wherein a two-piece capsule is filled in a first process module, and the filled two-piece capsule is merged with a half capsule, a second capsule or a tablet filled in a second process module.

36. The process as claimed in claim 33, including the step of separating the two or more pharmaceutical formulations by a physical barrier.

37. The process as claimed in claim 33, wherein the process modules are interchangeable.
